(19) United States
(12) Patent Application Publication
(10) Pub. No.: US 2024/0173191 A1
(43) Pub. Date: May 30, 2024

(54) TRAINING DEVICE FOR CARRYING OUT MUSCLE TRAINING FOR A MUSCLE

(71) Applicant: HEADIS GMBH, Weselberg (DE)

(72) Inventor: RENE WEGNER, Weselberg (DE)

(73) Assignee: HEADIS GMBH, WESELBERG (DE)

(21) Appl. No.: 18/282,998

(22) PCT Filed: Mar. 22, 2022
(86) PCT No.: PCT/DE2022/200053

§ 371 Date: Sep. 20, 2023

(30) Foreign Application Priority Data
Mar. 23, 2021 (DE) 10 2021 107 223.9

(51) Int. Cl. A61H 1/00; A61H 23/02; A61N 1/36

(52) U.S. Cl. CPC A61H 1/005; A61H 23/02; A61H 2201/0149; A61H 2201/10; A61H 2201/501; A61H 2201/5043; A61H 2201/5074; A61N 1/36003; A61N 1/36014

(57) ABSTRACT

A training device for carrying out a muscle training for a person's muscle. The training device includes a control unit having a signal unit, a main body, and a load measuring device arranged on the main body. The person's muscle contacts the load measuring device in use. The load measuring device has a load sensor which determines a load exerted by the person on the load measuring device at a measuring point which is correlated with a partial region of the muscle at a measuring point position. The control unit outputs a contraction signal to the person via the signal unit so that the person performs a signaled muscle contraction based thereon. A successful performance of the signaled muscle contraction is verifiable by comparing a load profile, which is determined by the load measuring device, with a comparison load profile.

### CROSS REFERENCE TO PRIOR APPLICATIONS

This application is a U.S. National Phase application under 35 U.S.C. § 371 of International Application No. PCT/DE2022/200053, filed on Mar. 22, 2022 and which claims benefit to German Patent Application No. 10 2021 107 223.9, filed on Mar. 23, 2021. The International Application was published in German on Sep. 29, 2022 as WO 2022/199766 A1 under PCT Article 21(2).

### FIELD

The present invention relates to a training device for carrying out muscle training of a muscle, in particular a specific muscle, of a person, the training device comprising a control unit, a main body, and a load measuring device arranged on the main body, wherein the muscle, in particular the specific muscle, is in contact with the load measuring device in a use position of the person, and the load measuring device comprises a load sensor or a plurality of load sensors for determining a load exerted by the person on the load measuring device at a respective measuring point correlated with a partial region of the muscle, at a respective measuring point position.

### BACKGROUND

Training devices of the type mentioned have previously been described so that a sitting position of a person sitting on a chair can, for example, be detected by corresponding load sensors. Load sensors in, for example, seating furniture or other devices are also in principle known. Interactive training is not, however, possible with corresponding devices.

### SUMMARY

An aspect of the present invention is to improve on the prior art.

In an embodiment, the present invention provides a training device for carrying out a muscle training for a muscle of a person. The training device includes a control unit which comprises a signal unit, a main body, and a load measuring device which is arranged on the main body. In a use position of the person, the muscle of the person is in contact with the load measuring device. The load measuring device comprises at least one load sensor which is configured to determine a load exerted by the person on the load measuring device at a measuring point which is correlated with a partial region of the muscle at a measuring point position, respectively. The control unit is configured so that a contraction signal can be output to the person via the signal unit so that the person performs a signaled muscle contraction based on the contraction signal. A successful performance of the signaled muscle contraction is verifiable by comparing a load profile, which is determined by the load measuring device, with a comparison load profile, in that, when the load profile and the comparison load profile correspond, a positive success signal is output, and, when the load profile and the comparison load profile do not correspond, a negative success signal is output.

### BRIEF DESCRIPTION OF THE DRAWINGS

The present invention is described in greater detail below on the basis of embodiments and of the drawings in which:

FIG. 1 is a schematic, isometric view of a workstation, comprising an integrated training device;

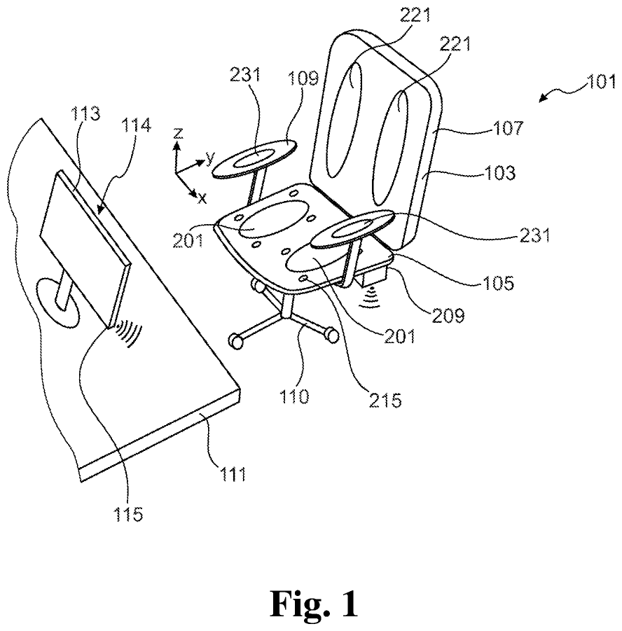

FIG. 2 is a schematic, isometric view of a sensor mat for measuring loads occurring on the sensor mat; and

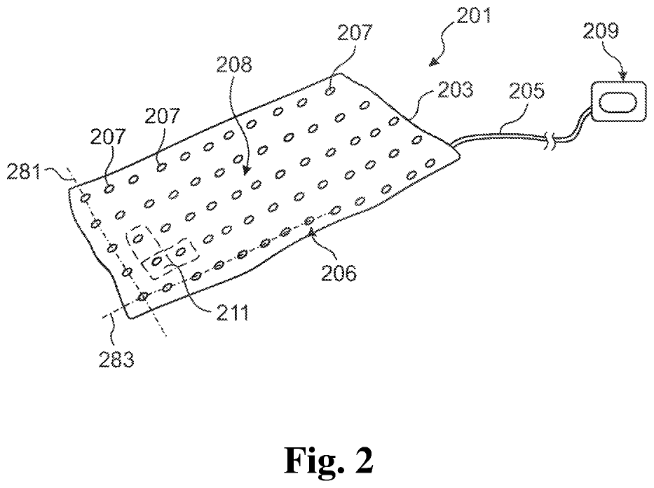

FIG. 3 is a three-dimensional graph for illustrating corresponding sensor signals of sensor mats.

### DETAILED DESCRIPTION

The present invention provides a training device for carrying out muscle training for a muscle, in particular for a specific muscle, of a person, comprising a control unit, a main body, and a load measuring device arranged on the main body, wherein the muscle, in particular the specific muscle, is in contact with the load measuring device in a use position of the person, and the load measuring device comprises a load sensor or a plurality of load sensors for determining a load exerted by the person on the load measuring device at a measuring point correlated with a partial region of the muscle, at a respective measuring point position, wherein the control unit comprises a signal unit, and the control unit is designed so that a contraction signal can be output to the person by the signal unit, so that the person carries out a signaled muscle contraction on the basis of the contraction signal, and successful performance of the signaled muscle contraction can be checked by a comparison of a load profile determined by the load measuring device with a comparison load profile, in that, when the load profile and the comparison load profile correspond, a positive success signal is output, and when the load profile and the comparison load profile do not correspond, a negative success signal is output.

In interaction with the control unit, the signal unit and the load measuring device, interactive action with the training device can thus take place, for example, in that the person receives, via the signal unit, a signal for carrying out a specific muscle contraction, and, in the case of the muscle contraction being carried out successfully, receives a positive success signal, for example, positive feedback via the signal unit, and thus the training success can be directly identified and, for example, also logged in the long term. In the reverse case, for example, in the case of contraction of the corresponding muscle that is not yet sufficient, a negative success signal would be output so that, for example, a further muscle contraction is prompted.

The following terms are explained in this connection:

A “training device” is a technical device, in particular an interactive device, via which a person can train a specific muscle, wherein the training device comprises, for example, mechanical components, sensor systems, and a corresponding control unit, and thus can also provide feedback to the person. In this case, “carrying out muscle training” describes the interactive contraction of a muscle, in particular a specific muscle, as a result of which, for example, a therapeutic effect for the muscle, a strengthening of the muscle, or a long-term relaxation of the muscle, is achieved by a correspondingly guided and controlled short-term contraction of the corresponding muscle. In this case, a “muscle” is a contractible organ of a living being and in this connection is to be understood not only as an individual muscle, but rather also interpreted broadly as a muscle group which is, for example, representative for a specific movement or a specific training goal.

In this connection, a “specific muscle” denotes that a defined muscle, for example, that is to undergo training in the context of sports medicine, or, analogously thereto, a corresponding muscle group, is detected correspondingly via the load sensors, isolated from surrounding muscle or also surrounding muscle groups, for example, and can subsequently be trained in a targeted manner so that, for example, in the context of muscle training, physiotherapy treatment, or also diagnosis in the context of sports medicine, the corresponding muscle contraction of the specific muscle can be carried out and evaluated. The specific muscle is thus, for example, a flexor which is located, for example, on a rear side of a thigh. A “person” is, for example, a human, wherein the training device can be used not only for humans, but rather also for a plurality of living beings having corresponding muscles, i.e., for example, also for the corresponding training of animals.

A “control unit” can in particular be an electronic unit and/or a computer which is designed to record corresponding signals, generate corresponding results therefrom, for example, via an algorithm, and output the results so that corresponding technical units are controlled, actuated, or influenced, for example, via an output.

A “main body” is, for example, a frame, a base frame, or another mechanical component, which holds essential components of the training device, in particular in a geometric relationship. A main body of the training device can thus, for example, be the frame of a chair, but the main body can also be designed to be flexible, non-rigid, or similar, and be formed, for example, by a fabric layer in the form of a mat or a sports underlay.

A “load measuring device” can be a technical, mechanical, or also electromechanical or electronic device, via which a load can be received and processed using measurement technology. In the simplest case, a load measuring device of this kind is in this case a mechanically translated weighing device, wherein electronic embodiments for determination and electronic conversion of corresponding loads can also be denoted thereby. A “load profile” is then recorded via the load measuring devices, wherein in this case, the load profile describes a, for example, local distribution of different loads or a local change in different loads along one dimension, two dimensions or also three spatial dimensions, in particular also depending on a time curve. It is noted with respect thereto that the respective terms in connection with the term “load” can in this case, in the simplest case, denote a load in the sense of a force, a pressure, but also any variable derived therefrom, such as a contact determination or a speed of change or change of a contact, a force or a pressure or another load variable, which is or are in each case significant for the muscle contraction.

A “use position” of the person is the position which the person or the living being assumes for expedient use of the training device. On a chair, this use position would, for example, be sitting upright, on a mat it would corresponding be lying flat. The use position is assumed to be analogous thereto for other training devices.

A “contact” describes the physical contact of the respective muscle with the load measuring device at a specific point or, for example, over a specific surface, so that, for example, the person lies or sits on the load measuring device or is brought into contact and/or held in contact with the load measuring device in another form.

The load measuring device in this case comprises various “load sensors” or also just one load sensor, which can be designed as a mechanical, an electromechanical, or also an electronic sensor, and can detect a corresponding “load” according to the description above and forward the detected load, for example, in the form of a mechanical or also an electronic signal. For this purpose, a corresponding “partial region” of the muscle, i.e., a specific portion of a muscle, is a part of the muscle significant in sport medicine, for example, or a volumetrically significant part of the muscle, for example, which may be characteristic for a muscle contraction, in contact with the load measuring device. Such a respective partial region of the muscle is in contact with a respectively correlated “measuring point”, i.e., a corresponding measuring location or a measuring site which is arranged at a respective “measuring point position” within or on the load measuring device. There is thus a relationship between the respective measuring point and the associated measuring point position, to the respective muscle or to the respective partial region of the muscle on the load measuring device, as a result of which a traceable, geometrically specified and/or reproducible location for the respective load can be detected.

A “signal unit” is a technical unit, for example, an electrical unit or also an electronic unit, which is suitable for outputting a corresponding signal in the form of a “contraction signal”, i.e., in the form of an instruction to carry out a muscle contraction, to the person. The signal unit is, for example, a lamp, a screen display, or, for example, also an acoustic signal.

A contraction signal can thus be output by the signal unit to the person so that the person then carries out a “signaled muscle contraction”, i.e., a provided muscle contraction that is to be carried out on the basis of the contraction signal. “Successful carrying out” is achieved, for example, if a sufficient load is exerted on the load measuring device via the signaled muscle contraction.

For this purpose, a “comparison” is carried out, which then outputs a corresponding determined load profile when the load profile and the comparison load profile “correspond”, i.e., in the event of a correspondence of the load profile with the comparison load profile that is sufficient according to the aim to be achieved, and a “positive success signal” is output. A positive success signal of this kind is, for example, an acoustic, optical or text output which is to be interpreted correspondingly and which is initiated via the control unit. In contrast thereto, a “non-correspondence” describes the situation where the load profile and the comparison load profile corresponding to the desired training success deviate from one another to such an extent that the training success is no longer provided. The output of a “negative success signal” in this case then takes place, which signal is output, for example, in the form of an acoustic warning signal, a text output for repeating the corresponding signaled muscle contraction, or similar.

In order to design the training device to be particularly user-friendly, the signal unit comprises an optical display unit, in particular a screen, so that an optical contraction signal can be output to the person via the optical display unit.

In this case, an “optical display unit” of this kind is designed so that an “optical contraction signal” can be output to the person, which signal can be perceived by eye, and thus functionally independently of corresponding physical movements, i.e., a signal perceptible by eye, comprising the instruction to carry out the signaled muscle contraction.

In an embodiment of the present invention, the signal unit can, for example, comprise a mechanical, in particular an electromechanical, actuator, wherein a mechanical contraction signal and/or a mechanical success signal can be output to the person via the mechanical actuator. An “actuator” which can output a “mechanical contraction signal”, for example, in the form of vibrations, pressure pulses or a single pressure, can thus, for example, directly cause the person to carry out the contraction of a corresponding muscle, for example, also arranged on the actuator. A “mechanical success signal” can in this case be designed, for example, in the form of a slight vibration so that the person receives a signal, via the slight vibration, for example, that the muscle contraction has been successfully carried out.

In order to also be able to carry out an active stimulation of a muscle contraction via the training device, the signal unit comprises an electrostimulator, wherein the electrostimulator enables an electromyostimulation of the muscle. An “electrostimulator” consists, for example, of a plurality of electrodes arranged on the main body, which electrodes are in contact with the corresponding muscle or the corresponding muscle group, and emit an electrical signal, via which what is known as an “electromyostimulation”, i.e., electrical excitation of the muscle contraction from outside the body, can be carried out.

In an embodiment of the present invention, the load sensor is or the load sensors are a pressure sensor or pressure sensors, wherein the pressure sensor or the pressure sensors is or are designed as a switching pressure sensor or switching pressure sensors, as a capacitive pressure sensor or capacitive pressure sensors, and/or a resistive pressure sensor or resistive pressure sensors, and/or a plurality of load sensors is arranged, in particular along a first main direction and/or a second main direction, in a matrix-like arrangement, on the load measuring device.

A muscle contraction can thus be detected via corresponding forces or also measured or derived pressures on the load measuring device. If the arrangement along a first main direction and along a second main direction is carried out in a matrix-like manner, it is then possible, for example, for a geometrically defined network of pressure sensors or other load sensors to be created, as a result of which the evaluation of the muscle contraction at different measuring points is greatly simplified.

A “pressure sensor” can be a measuring element for determining a pressure, i.e., for determining a force per surface area, wherein in this case a pressure sensor of this kind can also be designed in the form of a force sensor. It is thus possible for a pressure, i.e., the measured force relative to the respective influence surface, to be concluded, for example, from the measuring point position and a corresponding influence surface, assigned to the associated measuring point, for the force sensor. Such a pressure sensor of any design can therefore also be referred to synonymously as a force sensor, and also function as such. A “switching” pressure sensor or switching force sensor can generate a switching signal in the form of 0/1 information, for example, in the case of a limit force or a limit pressure being exceeded so that, for example, the loading of the corresponding force sensor or pressure sensor is detected. If, for example, a capacitive pressure sensor and/or a resistive pressure sensor or another suitable sensor is used, it is then possible to also qualitatively determine the magnitude of a corresponding pressure or the magnitude of a corresponding force on the respective measuring point.

In this connection, a “main direction” is, for example, a respective axis of a coordinate system, wherein here both cartesian coordinates and also polar coordinates or another expedient arrangement can be used in order to arrange corresponding load sensors in a matrix-like manner. Specifically adapted models or arrangements, for example, also adapted in a curved manner to a muscle profile, are here also expedient.

In order to be able to carry out a detailed evaluation with regard to the signaled muscle contraction, the control unit is designed to determine a load surface from the measuring point position of a plurality of measuring points, a load intensity at a respective measuring point, and/or a load volume, via a superimposition, in particular via a multiplication and/or a weighted multiplication of the load surface with the respective load intensity at the respective measuring point.

A “load surface” is, for example, the entirety, in terms of surface area, of those measuring points which, for example, exhibit a load change or which, provided that the comparison load profile corresponds, for example, to a rest position of the load measuring device, exhibit a measurable load compared therewith, for example, above a limit value for eliminating measuring inaccuracies. A determination in the form of a contact surface of the respective muscle analogously to one acting by muscle contraction is thus possible via the load measuring device so that, for example, a length, a width, or also a surface area, for example, in [cm²], can be used to make a determination regarding the strength or also the form of the muscle contraction. In this case, a “length” can, for example, be determined along a main direction of the muscle, a “width” correspondingly orthogonal thereto, and a “surface area”, for example, as a surface integral, an interval summation, or a numerical summation of the corresponding measuring points over the surface or also surface-like spatial dimensions of the measuring point positions, the latter, for example, in the case of a slightly curved contact surface in contact with the muscle. A “shape”, i.e., a geometry of the load surface, can be determined analogously thereto which, for example, allows a determination to be made regarding the muscle contraction in specific partial regions of the muscle in order to evaluate a training success.

A “load intensity” describes a signal which can provide information regarding a strength of the respective signal, i.e., is, for example, scaled between a basic load of “0” and a maximum load of “1”, or, for example, also in the form of a force in Newtons on a scale appropriate to the measurement purpose, so that a corresponding load intensity at the respective measuring positions results in an amplitude-based load profile.

In order to be able to make an even more accurate determination regarding the overall strength of the muscle contraction, a superimposition intensity of the muscle contraction is determined from a superimposition of the determined load surface with the determined load intensities of the muscle contraction at the respective measuring points, wherein the superimposition is formed in particular via a multiplication and/or a weighted multiplication of the load surface with the respective load intensity at the respective measuring point, and/or with integration of the load intensity over the load surface, so as to determine a load volume.

In this case, a “superimposition” of the determined load surface with the determined load intensities describes a mathematical operation taking into account a plurality of dimensions, for example, the spatial dimensions of the load or also a temporal dimension of the load at specific measuring points at a specific point in time, of the corresponding load of the muscle contraction. For example, a multiplication of the load surface with the respective, in particular amplitude-based, load intensity at the respective measuring point is thus performed, or the load intensity over the load surface is calculated in a mathematically continuous manner in the form of a integration, or also a weighted multiplication is performed, in which, for example, specific regions, specifically, for example, partial regions of the muscle that are particularly relevant in sports medicine, are taken into account separately or, for example, to a greater extent at their contact surface with the load measuring device. An integration in the sense of forming a mathematical integral of the load intensity over the load surface can in this case also be performed so that, for example, respective partial integrals are formed over specific regions of the load surface, or the superimposition is determined via summation of discrete calculations at respective measuring points, in the form of a numerical calculation, as a sufficiently accurate approximation of the mathematically exact integral. The result thereof is then, for example, a sum-like numerical value formed from a matrix having individual load values at specific measuring points, which numerical value is characteristic for the contraction carried out by the muscle. An integration or summation of the measured values over time can alternatively or also additionally take place so that, for example, the work achieved in the sense of training performance of the respective muscle over a time selected for training can be determined, and can be used for feedback regarding the training success.

In this case, a “load volume” is the measured variable which takes into account, as the numerical value, both the surface area, specifically the load surface, and also a corresponding local load intensity, its distribution, or the like, and adds the individual load intensities over the load surface analogously to a summation, so that the load volume is characteristic, for example, for the overall work achieved by the muscle at the respective measuring timepoint.

It is noted in connection therewith that the multiplication, the integration or another mathematical operation for the present invention can be carried out both in a mathematically exact manner from correspondingly derived functions, and also numerically, in an interval-switched manner, or in a similar manner, wherein, in this case, the respective manner of performing this can take into account a corresponding grid or corresponding spacings of measuring points, in the respective arrangement of the measuring point positions or the like. According to the present invention, in this case not only the mathematical precision of the determination of the load at the respective measuring point, but rather in particular the overall image, in terms of measuring technology, of the corresponding muscle contraction on the load measuring device, is decisive in order to be able to provide corresponding feedback on the strength, form, and quality of the muscle contraction with respect to training success.

In an embodiment of the present invention, the control unit comprises a smartphone, a tablet computer, and/or a personal computer. Via such electronic devices, which are typically present, for example, in an office or in a home, the control unit can, for example, also be implemented in the form of software on the corresponding electronic device so that, for example, in an office, a correspondingly equipped chair, as a training device, can be connected with a corresponding personal computer present at the office workstation, so that the training device is formed by the provided computer and a specially designed office chair.

The training device in this case in particular comprises a chair, an armchair, and/or a couch. The training device can in this case also be formed substantially by the chair, the armchair and/or the couch, having a corresponding associated control unit. In an embodiment, the load measuring device is in this case arranged in a seat surface, in a backrest, in a footrest, in an armrest, in a headrest and/or in a neck support of the chair or the armchair, and/or in a lying surface of the couch.

Corresponding muscles or also muscle groups, which are in contact, for example, with the backrest, a footrest, or an armrest, in a headrest and/or in a neck support of the chair or the armchair, or a corresponding lying surface of the couch, can thus be trained in a targeted manner.

The training device can also comprise a mat, a sports mat, a yoga mat, an item of clothing, or the like.

The present invention will be explained in greater detail below with reference to embodiments as shown in the drawings.

A workstation 101 comprises an office chair 103 having a seat surface 105, a backrest 107, and two armrests 109. The office chair can furthermore comprise a headrest and/or a neck support (not shown in the present example). The office chair 103 furthermore comprises a frame 110. The office chair 103 is at a desk 111 and comprises sensor mats 201 in its seat surface 105, sensor mats 221 in the backrest 107, and sensor mats 231 in the armrests 109 for detecting pressures acting on the respective components. By way of example, actuators 215 are show in the seat surface 105. A computer 113 comprising a screen 114 is arranged on the desk 111, which computer 113 is designed as a conventional desktop PC and is typically used for working at the workstation 101.

The computer 113 comprises a communication module 115 (shown schematically) which, in the example shown, is a Bluetooth module. The communication module 115 can thus both send and receive signals. The office chair 103 also comprises a communication module 209 which can exchange data with the communication module 115 via the Bluetooth protocol.

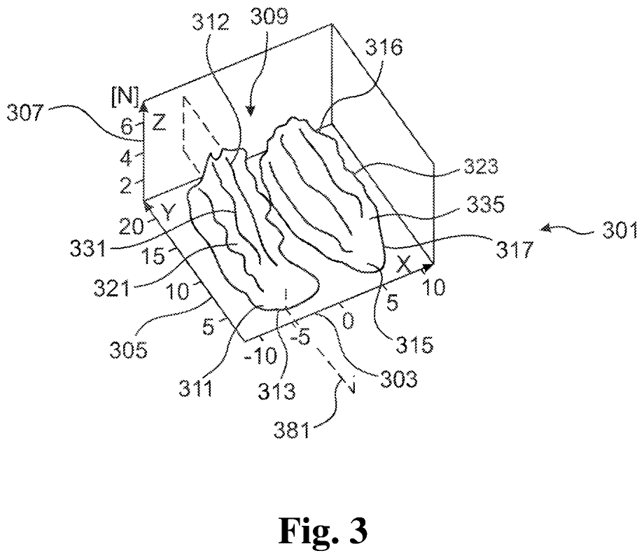

A sensor mat 201 described by way of example, having a function analogous to the sensor mats 221 and sensor mats 231, comprises a mat body 203 consisting of a woven fabric. The mat body 203 is connected to the communication module 209 via a cable 205. A plurality of pressure sensors 207 is arranged inside the mat body 203, within a matrix field 208, along transverse axes 281 and along longitudinal axes 283, which pressure sensors 207 are designed in technical terms as force sensors. The correlation of force to pressure is in this case formed from a measured force at a respective measuring point 206, and a respective reference surface 211, in which the measured force is based on the respective reference surface 211, so that a pressure can be output at the respective measuring point 206. Reference will in the following therefore be made simply to “pressure” in the relevant context even if, technically, a force measurement is performed. The pressure sensors 207 are resistive pressure sensors and can, as described above, detect a local pressure, acting orthogonally on the mat body 203, at a relevant measuring point 206. For each measuring point 206, a corresponding pressure load on the mat body 203 can thus be concluded from the arrangement in the matrix field 208. This results, analogously to the respective pressure load, in a respective measured value. The communication module 209 in this case serves to record the measured values conducted from the pressure sensors 207 via the cable 205, in the form of pressure signals, and to forward these to the communication module 115 of the computer 113, wherein, for example, a pressure map (see also FIG. 3) can then be created via the computer 113, or also corresponding tabular recording of sensor signals of the pressure sensors 207 over corresponding time portions or in time intervals is made possible. In the present example, the communication module 209 records each signal of the pressure sensors 207 at a frequency of 60 Hz, i.e., 60 measurements per second, and forwards these to the computer 113.

The sensor mat 201 records corresponding pressure data on either side of the seat surface 105 of a person sitting on the office chair 103, and specifically the pressure of the muscle in contact with the sensor mat 201 in each case. This is, in the present case, by way of example, the “m. gluteus maximus”, i.e., the large muscle in the buttocks, and the “m. semitendinosus”, i.e., one of the rear flexor muscles of the thigh. In this case, in the rest position, a specific measurement image results, i.e., a pressure distribution from person's own weight, wherein the person then subsequently tenses corresponding muscles for a measurement. By way of example, for this, a corresponding function 311 and a function 315 for the respective region of the buttocks and the respective thigh, during tensing of the muscles, at a specific timepoint of the measurement, is shown in a graph 301.

The graph 301 has an x-coordinate 303 which represents a direction transverse to the seat surface 105 of the office chair 103. Orthogonally thereto, the graph 301 has a y-coordinate 305 which extends on the seat surface 105 analogously to the seat depth. A z-coordinate 307 is in turn positioned orthogonally thereto so that a three-dimensional cartesian coordinate system is spanned by a coordinate space 309. In this case, the x-coordinate 303 and the y-coordinate 305 span a plane in parallel with the seat surface 105, the z-coordinate 307 constitutes an axis for representing a pressure on the sensor mat 201, and specifically at the respective measuring point 206 in the plane of the x-coordinate 303 and the y-coordinate 305.

In the coordinate space 309, a function 311 for imaging the buttocks region and the thigh of the person on one side of the body, and a function 315 for imaging the muscle groups on the other side of the body, are shown. The function 311 has various measured values 331 and a maximum point 312, i.e., the point of maximum pressure, and, analogously thereto, the function 315 has various measured values 335 and a maximum point 316. The function 311 and the function 315 likewise form a base area 313 and a base area 317, via which a corresponding pressure region on the seat surface 105 is described. By tensing the muscles, for example, in the back thigh region, the person thus exerts a pressure on the seat surface 105 which is non-stationary with respect to the rest position, which is detected by the sensor mats 201 and processed by the computer 113. In this case, a respective volume under the respective function can also be determined from the x-y plane formed from the x-coordinate 303 and the y-coordinate 305 in order to derive therefrom the achieved work of the muscle.

The actuators 215, which can also be present analogously thereto and having the same effect in the backrest 107 and in the armrests 109, are designed as piezo actuators and can, actuated by the computer 113 via the communication modules 115 and 209, emit vibration signals of different intensities and frequencies, and thus output these to a person sitting on the office chair 103.

The workstation 101 can thus be used, equipped in the described manner, as a training device, for example, when corresponding muscle groups become fatigued from sitting at the desk 111 for a long time, or, for example, when a corresponding time period, risking fatigue of precisely these muscles, has elapsed. A software that serves for operating the training device is installed on the computer 113, which thus simultaneously makes the computer 113 into a control unit for the office chair 103. Firstly, a corresponding load profile is recorded when the person is at rest, analogously to the function 311 and the function 315, wherein this takes place via the sensor mat 201 in the seat surface 105. At the same time, a corresponding recording of the rest signals of corresponding muscles is carried out via the analogously designed sensor mat 221 in the backrest 107 and via the analogously designed, but significantly smaller, sensor mats 231 in the armrests 109.

On the basis of the example of the seat surface 105, a corresponding stimulation and excitation to muscle contraction, as training, will now be explained:

The actuators 215 are, for example, after completion of a motionless phase detected via the sensor mats 201, actuated and set into vibration, via the software on the computer 113, via the transmission paths formed between the communication module 115 and the communication module 209, so that the person (not shown) sitting on the chair can contract the corresponding muscle groups arranged on the back of the thigh, prompted by the vibration. Provided that the sensor mats 201 detect a sufficient muscle contraction via a comparison of the functions 311 and 315 with corresponding signals in the rest position, or a predetermined contraction duration is achieved, the actuators 215 output a slight signal, which is significantly lower compared with the signal stimulating muscle contraction, in the form of a slight vibration, so that the person sitting on the chair is informed of the success or the end of the corresponding exercise. Analogously thereto, an optical display on the screen 114 and/or an acoustic display can take place. If the corresponding muscle contraction is not carried out satisfactorily, the stimulation via the actuators 215 can, for example, be repeated so that the muscle contraction is then carried out. Alternatively or also in addition, an electrostimulator (not shown) can also be arranged, for example, in the seat surface 105, which carries out an electromyostimulation via electrical pulses, and thus assists or triggers the muscle contraction.

Analogously thereto, for example, via the sensor mats 221 and corresponding actuators (not shown) in the backrest 107, the stimulation of the back muscles, for example, by an initiated back movement, or, analogously thereto, the stimulation of the forearm muscles in conjunction with the sensor mats 231 in the armrests 109 and corresponding actuators (not shown), initiate a display or an equivalent signal on the screen 114.

The present invention is not limited to embodiments described herein; reference should be had to the appended claims.

### LIST OF REFERENCE NUMERALS

101 workstation
103 office chair
105 seat surface
107 backrest
109 armrest
110 frame
111 desk
113 computer
114 screen
115 communication module
201 sensor mat
203 mat body
205 cable
206 measuring point
207 pressure sensor
208 matrix field
209 communication module
211 reference surface
215 actuator
221 sensor mat
231 sensor mat
281 transverse axis
283 longitudinal axis
301 graph
303 x-coordinate
305 y-coordinate
307 z-coordinate
309 coordinate space
311 function
312 maximum point
313 base area
315 function
316 maximum point
317 base area
321 pressure volume
323 pressure volume
331 measured value
335 measured value
381 sectional plane

What is claimed is:

1-9. (canceled)

10. A training device for carrying out a muscle training for a muscle of a person, the training device comprising:
a control unit which comprises a signal unit;
a main body; and
a load measuring device which is arranged on the main body, wherein, in a use position of the person, the muscle of the person is in contact with the load measuring device, the load measuring device comprising at least one load sensor which is configured to determine a load exerted by the person on the load measuring device at a measuring point which is correlated with a partial region of the muscle at a measuring point position, respectively,
wherein,
the control unit is configured so that a contraction signal can be output to the person via the signal unit so that the person performs a signaled muscle contraction based on the contraction signal, and
a successful performance of the signaled muscle contraction is verifiable by comparing a load profile, which is determined by the load measuring device, with a comparison load profile, in that, when the load profile and the comparison load profile correspond, a positive success signal is output, and, when the load profile and the comparison load profile do not correspond, a negative success signal is output.

11. The training device as recited in claim 10, wherein the muscle is a specific muscle of the person.

12. The training device as recited in claim 10, wherein the signal unit comprises an optical display unit which is configured to output an optical contraction signal to the person.

13. The training device as recited in claim 12, wherein the optical display unit is a screen.

14. The training device as recited in claim 12, wherein the signal unit further comprises a mechanical actuator which is configured to output at least one of a mechanical contraction signal and a mechanical success signal to the person.

15. The training device as recited in claim 14, wherein the mechanical actuator is an electromechanical actuator.

16. The training device as recited in claim 14, wherein the signal unit further comprises an electrostimulator which is configured to enable an electromyostimulation of the muscle.

17. The training device as recited in claim 10, wherein,
the at least one load sensor is a pressure sensor, and
the pressure sensor is designed as at least one of a switching pressure sensor, a capacitive pressure sensor, and a resistive pressure sensor.

18. The training device as recited in claim 17, wherein,
the at least one load sensor provided as a plurality of load sensors, and
the plurality of load sensors is arranged in a matrix-like arrangement on the load measuring device.

19. The training device as recited in claim 18, wherein the plurality of load sensors is further arranged at least one of along a first main direction and a second main direction in the matrix-like arrangement on the load measuring device.

20. The training device as recited in claim 10, wherein the control unit is further configured to determine a load surface from the measuring point position of at least one of a plurality of measuring points, a load intensity at the respective measuring point, and a load volume, via a superimposition.

21. The training device as recited in claim 20, wherein the superimposition is at least one of a multiplication and a weighted multiplication of the load surface with the respective load intensity at the respective measuring point.

22. The training device as recited in claim 10, wherein the control unit comprises at least one of a smartphone, a tablet computer, and a personal computer.

23. The training device as recited in claim 10, further comprising at least one of:
a chair,
an armchair, and
a couch.

24. The training device as recited in claim 23, wherein
the chair and the armchair each comprise a seat surface, a backrest, a footrest, an armrest, a headrest, and a neck support,
the couch comprises a lying surface, and
the load measuring device is arranged in at least one of,
in at least one of the seat surface, the backrest, the footrest, the armrest, the headrest, and the neck support of the chair or of the armchair, and
in the lying surface of the couch.
